(19) United States
(12) Patent Application Publication
(10) Pub. No.: US 2022/0063632 A1
(43) Pub. Date: Mar. 3, 2022

(54) OPERATOR HEALTH MONITORING SYSTEM

(71) Applicant: DEERE & COMPANY, Moline, IL (US)

(72) Inventors: Brian M. Huenink, Cedar Grove, WI (US); Gregory O. Mcconoughey, Le Claire, IA (US)

(21) Appl. No.: 17/307,837

(22) Filed: May 4, 2021

Related U.S. Application Data
(60) Provisional application No. 63/072,769, filed on Aug. 31, 2020.

(51) Int. Cl. B60W 40/08; G16H 40/67; B60W 50/14

(52) U.S. Cl. CPC B60W 40/08; G16H 40/67; B60W 2050/143; B60W 2040/0872; B60W 50/14

(57) ABSTRACT

An operator health monitoring system includes a central monitoring station and one or more utility vehicles positioned within a working site. Each utility vehicle includes an operator cab and an operator support positioned within the operator cab. Each utility vehicle also includes a control system including a sensor operable to sense a health characteristic of an operator positioned on the operator support, and a controller including a processor and a memory. The controller is configured to receive a signal from the sensor representative of the health characteristic. The control system further includes a communications module configured to transmit information associated with the health characteristic from the controller to the central monitoring station via a network. The central monitoring station is configured to monitor the health characteristic of the operator of each utility vehicle of the one or more utility vehicles.

### CROSS-REFERENCE TO RELATED APPLICATIONS

This application claims priority to U.S. Provisional Patent Application No. 63/072,769 filed on Aug. 31, 2020, the entire contents of which is incorporated herein by reference.

### BACKGROUND

The present disclosure relates to an operator health monitoring system for operators of utility vehicles, in particular utility vehicles associated with agricultural equipment such as cotton harvesters and combine harvesters, and forestry and construction equipment such as skidders, loads, harvesters, and road paving equipment, mining equipment, etc. This equipment is often in operation in large fields or sites that may be sparsely populated.

### SUMMARY OF THE DISCLOSURE

The present disclosure relates to an operator health monitoring system for operators of utility vehicles, in particular utility vehicles associated with agricultural equipment such as cotton harvesters and combine harvesters, and forestry and construction equipment such as skidders, loads, harvesters, and road paving equipment, mining equipment, etc. This equipment is often in operation in large fields or sites that may be sparsely populated.

The present disclosure provides, in one aspect, an operator health monitoring system including a central monitoring station and one or more utility vehicles positioned within a working site. Each utility vehicle of the one or more utility vehicles includes a chassis, a prime mover supported by the chassis, an operator cab supported by the chassis, and an operator support positioned within the operator cab. The operator support includes a seat and a backrest coupled to the seat. Each utility vehicle also includes a control system including a sensor operable to sense a health characteristic of an operator positioned on the operator support, and a controller including a processor and a memory. The controller is configured to receive a signal from the sensor representative of the health characteristic. The control system further includes a communications module configured to transmit information associated with the health characteristic from the controller to the central monitoring station via a network. The central monitoring station is configured to monitor the health characteristic of the operator of each utility vehicle of the one or more utility vehicles.

The present disclosure provides, in another aspect, a monitoring station in electrical communication with one or more utility vehicles positioned within a working site. The monitoring station includes a control system including a controller having a processor and a memory. The controller is configured to receive a signal from a sensor operable to sense a health characteristic of an operator of one of the one or more utility vehicles. The controller is adapted to store and analyze the information associated with the health characteristic of the operator of the one of the one or more utility vehicles to monitor the health characteristic of the operator of the one of the one or more utility vehicles. The controller is configured to determine a working characteristic of the operator based on the transmitted health characteristic. The controller is configured to send a signal associated with adjusting a working parameter of the operator based on the working characteristic.

The present disclosure provides, in yet another aspect, an operator cab assembly for a utility vehicle. The operator cab assembly includes a housing and a chair positioned within the housing and configured to support an operator of the utility vehicle. The operator cab also includes a sensor assembly including one or more sensors operable to sense a health characteristic of the operator.

Other features and aspects of the disclosure will become apparent by consideration of the following detailed description and accompanying drawings.

### BRIEF DESCRIPTION OF THE DRAWINGS

FIG. 1 is a schematic view of an operator health monitoring system including a central monitoring station and a plurality of utility vehicles.

FIG. 2 is a perspective view of a utility vehicle including an operator cab.

FIG. 3 is a perspective view of an operator support positioned within the cab of the vehicle of FIG. 2.

FIG. 4 is a schematic of a control and communication system of the utility vehicle of FIG. 1.

FIG. 5 is an example of a schematic of a display screen for the operator health monitoring system of FIG. 1.

Before any embodiments of the disclosure are explained in detail, it is to be understood that the disclosure is not limited in its application to the details of construction and the arrangement of components set forth in the following description or illustrated in the following drawings. The disclosure is capable of supporting other embodiments and of being practiced or of being carried out in various ways. Also, it is to be understood that the phraseology and terminology used herein is for the purpose of description and should not be regarded as limiting.

### DETAILED DESCRIPTION

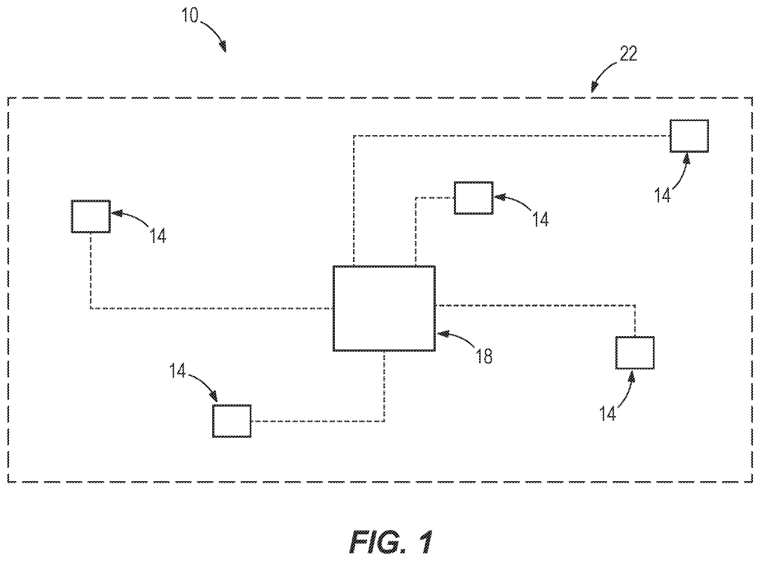

As shown in FIG. 1, an operator health monitoring system 10 includes a plurality of utility vehicles 14 in communication with a central monitoring station 18. The operator health monitoring system 10 is operable for a working site 22. More specifically, the working site 22 may be defined as an area in which the utility vehicles 14 are performing work. For example, the working site 22 may be a construction site, etc., with several operating utility vehicles 14, such as backhoe loaders. The working site 22 is typically a remote location (i.e., unpopulated area such as mine, forest, farming field, etc.). The operation of each utility vehicle 14 is locally controlled by an operator.

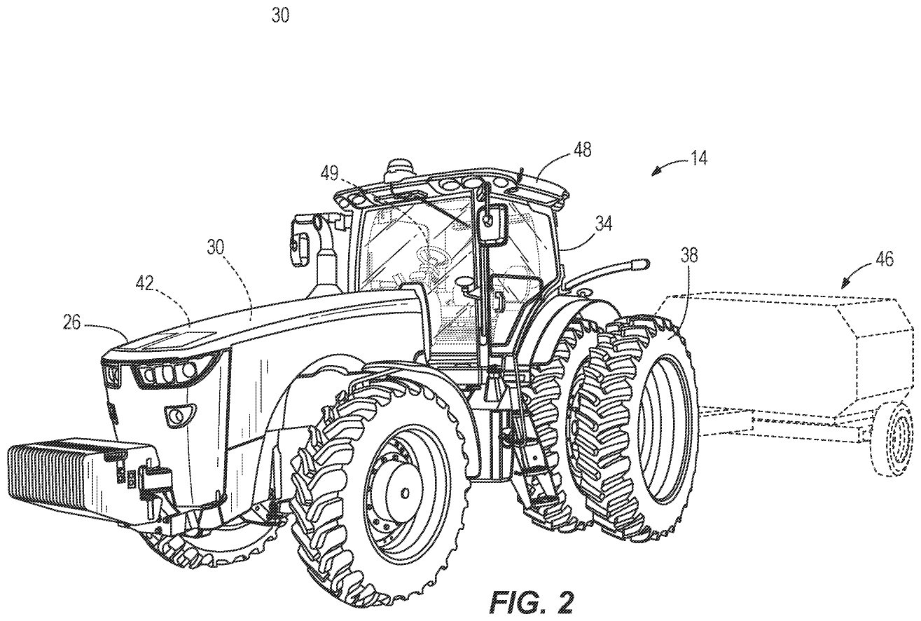

FIG. 2 illustrates one example of a utility vehicle 14. The utility vehicle 14 is in the form of a tractor, however, the utility vehicle 14 may be, for example, a harvester, crop sprayer, construction vehicle, forestry vehicle, mining vehicle, or other utility vehicle depending on the worksite 22. The utility vehicle 14 includes a chassis 26, a prime mover 30, an operator cab 34, a plurality of ground-engaging devices 38, and a control system 42.

The chassis 26 supports the prime mover 30 and the control system 42. The prime mover 30 is configured to move the utility vehicle 14 in a direction of travel via the ground engaging devices 38 and includes an engine, such as a diesel engine. The illustrated ground-engaging devices 38 are wheels, but tracks or other suitable ground-engaging devices can be utilized. The control system 42 includes a vehicle control unit (VCU).

In the illustrated embodiment, the utility vehicle 14 is coupled to and towing an implement 46 in the form of a planter coupled to a rear of the chassis 26. In other embodiments, the utility vehicle 14 may be coupled to any suitable implement 46 (e.g., a ripper, front end loader, bucket, manure spreaders, etc.), and the implement may be also coupled instead to the front of the chassis 26. Still in other embodiments, more than one implement 46 may be coupled to the chassis 26. For example, a first implement may be coupled to the front of the chassis 26 and a rear implement may be coupled to the rear of the chassis 26.

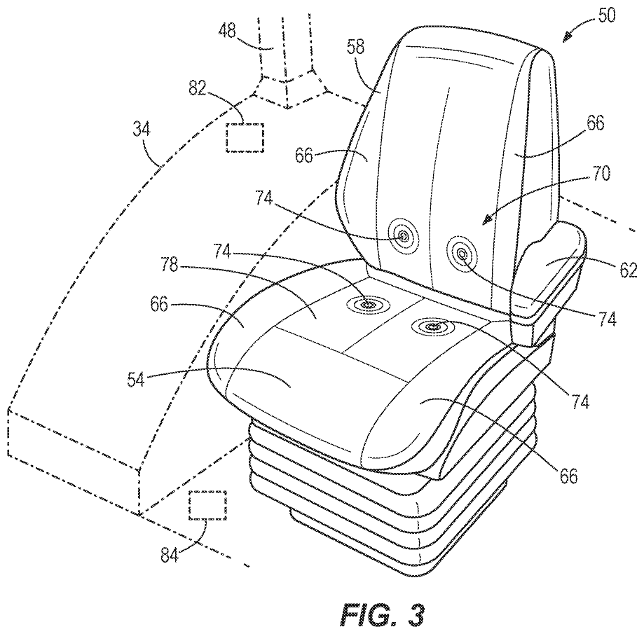

With reference to FIGS. 2 and 3, the cab 34 comprises a housing 48 and contains a vehicle operation system 49 and an operator support (e.g., chair 50).

The vehicle operation system 49 can include different combinations of input devices, such as a steering wheel, control levers, joysticks, control pedals, control buttons, control displays, and other input devices. The operator is typically seated in the chair 50 during use of the utility vehicle 14 and positioned to actuate one or more input devices of the vehicle operation system 49 for purposes of operating the utility vehicle 14 and the attached implement 46.

The housing 48 includes one or more walls to form the cab 34 such that an interior of the cab 34, including the chair 50, is not exposed to the environment.

The chair 50 includes a seat 54, a backrest 58 coupled to the seat 54 and positioned transversely thereto, and optionally one or more armrests 62 positioned on either or both sides of the seat 54. The seat 54 and backrest 58 may be made of a different materials and both generally include a frame (e.g., metal, rigid plastic, etc., not shown) that provides the general shape and support for the chair, a compressible material such as a foam (not shown) placed on the frame for cushion, comfort, and ergonomics, and a cover 78 (e.g., nylon, leather, etc.) that holds the compressible material relative to the frame. The seat 54 and/or the backrest 58 may include lateral cushion bolsters 66 on one or both sides of the seat 54 or backrest 58. The chair 50 supports the operator during operation.

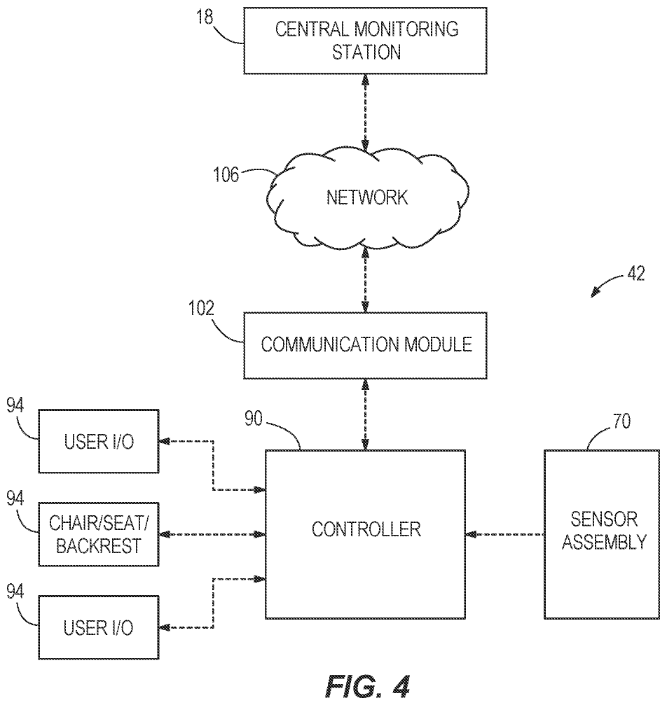

With reference to FIGS. 3 and 4, the cab 34 further includes a sensor assembly 70 comprising one or more sensors 74 formed with the chair 50 during manufacture or inserted into or otherwise coupled thereto afterward. The sensor assembly 70 can detect information relating to the health or functioning (referred to herein as health characteristics) of the seated operator. For example, as shown in FIG. 3, the chair 50 includes four sensors 74, two of which are positioned within the seat 54 and two of which are positioned within the backrest 58. The sensors 74 are positioned at a suitable depth relative to the cover 78, to include within the cushion layer, between the cushion layer and the cover 78, and/or at least partially on or flush with the cover 78 such that the sensor 74 is exposed and/or can directly contact the seated operator or have an unobstructed path to the seated operator.

The sensor assembly 70 may include one or more of a temperature sensor, a proximity sensor, a pressure sensor, oximetry sensor, blood pressure sensor, electrocardiogram (ECG) sensor, heart rate sensor, etc. One or more of the sensors 74 is used to detect a biometric or health characteristic (e.g., heart rate, respiratory rate, blood pressure, etc.) of the seated operator. In some embodiments, certain of the plurality of sensors 74 could be duplicative (i.e., to gather similar information from two or more seat positions, or as backup to another sensor) or each sensor 74 could serve a distinct purpose.

Although the sensors 74 primarily receive input from operator contact or temperature readings, sensors associated with the sensor assembly 70 may also receive information visually. In particular, the sensor assembly 70 can include one or more infrared sensors and/or cameras 82 positioned within or outside of the cab 34 to assess visual indicators of the operator's functioning or to identify any health-related conditions. For example, as shown in FIG. 3, the sensor assembly 70 includes a camera 82 supported by one of the walls of the cab 34. The camera 82 is oriented toward the chair 50 to obtain information as to the operator's overall positioning and in some instances, facial changes that could signal a condition. The camera 82, or the infrared sensor, can be operable to detect a body temperature of the seated operator.

Still further, the sensor assembly 70 can include one or more environmental sensors 84 positioned within or outside of the cab 34 to assess environmental factors surrounding the seated operator within or outside of the cab 34. The environmental sensors can include one or more of a temperature sensor, air quality sensor, moisture content sensor, etc., and can be used in conjunction with biometric data sensors.

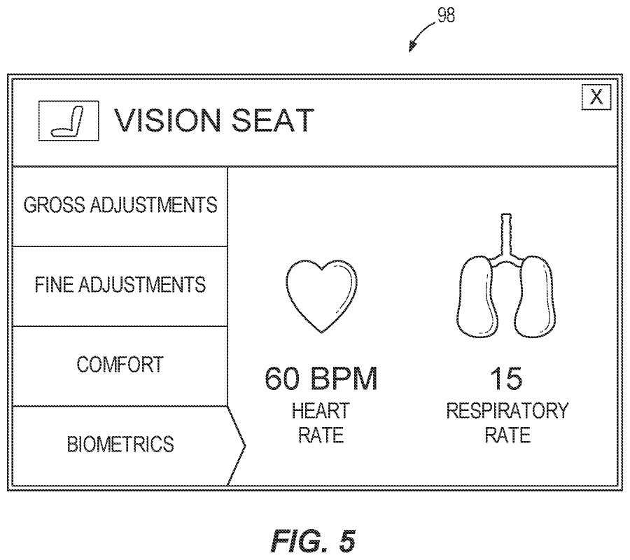

With reference to FIGS. 4 and 5, the control system 42 of the utility vehicle 14 includes a controller 90, a user input/output module 94, and a communications module 102.

The controller 90 is in electrical communication with the sensor assembly 70. The controller 90 receives information relating to the health or functioning of the seated operator from the sensor assembly 70. The controller 90 can include a memory and processor for determining the health characteristics of the seated operator in real time and over a period of time, with any modifications based on operator preference or requirements.

Communication between the controller 90 and the different components can be accomplished through a CAN bus, another communication link (e.g., wireless transceivers), or through a direct connection.

The module 94 has a plurality of inputs and outputs that are operable to receive and transmit information and commands to and from different components (e.g., sensors 74, 82, 84). The user input/output module 94 can also include one or more output devices such as, for example, a display device in the form of a control screen 98 (FIG. 5) located in the cab 34. The controller 90 may locally display, via the control screen 98, one or more health characteristics (e.g., heart rate, respiratory rate, bold pressure, etc.) detected by the sensor assembly 70 to the seated operator within the cab 34.

With particular reference to FIG. 5, the operator can locally review and/or monitor the one or more health characteristics detected by the sensor assembly 70, via the control screen 98. The operator can further use the control screen 98 to facilitate self-adjustments related to a specific health characteristic during operation of the utility vehicle 14. For example, the operator can visually determine the detected heart rate and respiratory rate, and the operator can determine, and/or the control screen itself 98 can indicate, that the operator is stressed or fatigued. Subsequently, the operator can use the displayed health characteristics to facilitate an operational adjustment related to a working parameter of the operator (e.g., the operator should take a break, be replaced by another operator, etc.).

The control system 42 further includes the communication module 102 configured to connect to and communicate with other devices (e.g., a computer, another work machine, etc.) through a network 106. The network 106 can be, for example, a wide area network (“WAN”) (e.g., a global positioning system (“GPS”), a TCP/IP based network, a cellular network, etc.).

The connections between the controller 90 and the communications module 102 may be wired connections, wireless connections, or a combination of wireless and wired connections. The connections between the communications module 102 and the network 106 are wireless to enable freedom of movement based on the operational location of the utility vehicle 14 in the working site 22.

If applicable, the controller 90 communicates with the central monitoring station 18 via the network 106. The central monitoring station 18, in some embodiments as shown in FIG. 1, is located within the area of the working site 22. Alternatively, the central monitoring station 18 is located outside of the area of the working site 22 (i.e., off-site). Due to the transmission of health-related information, or for other reasons, communications through the network 106 by the communications module 102 or the controller 90 can be protected using one or more encryption techniques, such as those techniques provided in the IEEE 802.1 standard for port-based network security, pre-shared key, Extensible Authentication Protocol (“EAP”), Wired Equivalency Privacy (“WEP”), Temporal Key Integrity Protocol (“TKIP”), Wi-Fi Protected Access (“WPA”), and the like.

The central monitoring station 18 include a centrally located computer, a network of computers, or one or more centrally located servers. The central monitoring station 18 can be adapted to store, interpret, analyze, and communicate data from one or more utility vehicles 14, and can also interpret and analyze the data and communicate the interpreted and analyzed data to a user (e.g., manager of the working site 22, etc.) by visual displays (e.g., similar to control screen 98, FIG. 5), noise alarms, etc.

In particular, the central monitoring station 18 receives data associated with the health characteristics of a seated operator from the controller 90 of each working utility vehicle 14 and monitors the health characteristics of each operator as the operator is controlling the operation of each respective utility vehicle 14 at the worksite 22. The central monitoring station 18 identifies a status of the operator of each respective utility vehicle 14 (e.g., comfortable, stressed, fatigued, emergency, etc.) to a user of the central monitoring station 18.

In addition, the central monitoring station 18 may determine a working characteristic of the operator of the respective utility vehicle 14 (e.g., operator is working efficiently, operator is working slowly, operator is not working, etc.) based on the data received.

In some embodiments, the central monitoring station 18 further stores the information associated with the health characteristics of the specific operator in relation to the work performed and can make comparisons over time or periodically. For example, the central monitoring station 18 is configured to determine and store an average heart rate of the operator based on the information associated with the health characteristic detected by the sensor assembly 70. The average heart rate for the specific operator may be compared to current information being transmitted to the central monitoring station 18 in order to determine the status and/or working characteristic of the operator. Accordingly, the central monitoring station 18 may be configured to track patterns of the status and working characteristic for the specific operator, and/or use the stored information to determine a current status or condition of the operator. In some circumstances, the central monitoring station 18 may alert a user (e.g., by a displace device 98, alarm, etc.) that the status and/or the working characteristic of the operator has reached a predetermined level based on any of the aforementioned determinations, health-related or otherwise. In certain situations, the central monitoring station 14 can alert emergency services as to a significant health-related event.

In yet other embodiments, a manager or supervisor with or at the central monitoring station 18 may determine, based on the information received and/or stored in the central monitoring station 14, that an operational adjustment should be made related to a working parameter of the operator in a utility vehicle 14 (e.g., stop continued working, check on the operator, operator should take a break, etc.). In one application, a user may replace the operator of the respective utility vehicle 14 prior to the status of the operator reaching a predetermined level (e.g., the operator is stressed, the operator is fatigued, etc.) that could affect performance.

Although the disclosure has been described in detail with reference to certain preferred embodiments, variations and modifications exist within the scope and spirit of one or more independent aspects of the disclosure as described.

Various features of the disclosure are set forth in the following claims.

1-20. (canceled)

21. An operator health monitoring system comprising:
a central monitoring station; and
one or more utility vehicles positioned within a working site, each utility vehicle of the one or more utility vehicles including
a chassis,
a prime mover supported by the chassis,
an operator cab supported by the chassis,
an operator support positioned within the operator cab, the operator support including a seat and a backrest coupled to the seat, and
a control system including
a sensor operable to sense a health characteristic of an operator positioned on the operator support,
a controller including a processor and a memory, the controller configured to receive a signal from the sensor representative of the health characteristic, and
a communications module configured to transmit information associated with the health characteristic from the controller to the central monitoring station via a network,
wherein the central monitoring station is configured to monitor the health characteristic of the operator of each utility vehicle of the one or more utility vehicles.

22. The operator health monitoring system of claim 21, wherein the sensor of each utility vehicle of the one or more utility vehicles is coupled to one of the seat, the backrest, and the operator cab.

23. The operator health monitoring system of claim 21, wherein at least one utility vehicle of the one or more utility vehicles further includes a display device located in the operator cab, and wherein the controller of the at least one utility vehicle of the one or more utility vehicles is configured to display, via the display device, the health characteristic of the operator positioned on the operator support of the at least one utility vehicle of the one or more utility vehicles.

24. The operator health monitoring system of claim 21, wherein the working site is a defined area in which the one or more utility vehicles are performing work, and wherein the central monitoring station is located outside of the defined area.

25. The operator health monitoring system of claim 21, wherein the central monitoring station includes a display device configured to display the information associated with the health characteristic of the operator of each utility vehicle of the one or more utility vehicles to a user of the central monitoring station.

26. The operator health monitoring system of claim 21, wherein the central monitoring station includes one or more computers adapted to store and analyze the information associated with the health characteristic of the operator of each utility vehicle of the one or more utility vehicles.

27. The operator health monitoring system of claim 21, wherein the central monitoring station is configured to determine one of a working characteristic and a status of the operator of one utility vehicle of the one or more utility vehicles.

28. The operator health monitoring system of claim 27, wherein the central monitoring station is configured to adjust a working parameter of the operator of the one utility vehicle of the one or more utility vehicles based on the one of the working characteristic and the status.

29. The operator health monitoring system of claim 21, wherein each utility vehicle of the one or more utility vehicles further includes a plurality of sensors supported by the chassis, wherein each sensor of the plurality of sensors is operable to sense a health characteristic of the operator positioned on the operator support of the respective utility vehicle, and wherein the controller of each utility vehicle is configured to receive a signal from each sensor of the plurality of sensors representative of the health characteristic.

30. A monitoring station in electrical communication with one or more utility vehicles positioned within a working site, the monitoring station comprising:
a control system including
a controller including a processor and a memory, the controller configured to receive a signal from a sensor operable to sense a health characteristic of an operator of one of the one or more utility vehicles;
wherein the controller is adapted to store and analyze the information associated with the health characteristic of the operator of the one of the one or more utility vehicles to monitor the health characteristic of the operator of the one of the one or more utility vehicles;
wherein the controller is configured to determine a working characteristic of the operator based on the transmitted health characteristic; and
wherein the controller is configured to send a signal associated with adjusting a working parameter of the operator based on the working characteristic.

31. The monitoring station of claim 30, further comprising a display device configured to display the information associated with the health characteristic of the operator of the one of the one or more utility vehicles to a user of the monitoring station.

32. The monitoring station of claim 30, wherein the working characteristic includes one of the operator of the one of the one or more utility vehicles working efficiently, working slowly, or not working.

33. The monitoring station of claim 30, wherein the working parameter is one of stopping continued working of the operator and taking a predetermined break period in work by the operator.

34. The monitoring station of claim 30, wherein the controller is also configured to determine a status of the operator of the one of the one or more utility vehicles based on the transmitted health characteristic, and wherein the controller is configured to send a signal associated with adjusting the working parameter of the operator based on the status.

35. The monitoring station of claim 34, wherein the status includes one of the operator of the one of the one or more utility vehicles is comfortable, stressed, fatigued, or emergency.

36. An operator cab assembly for a utility vehicle, the operator cab assembly comprising:
a housing;
a chair positioned within the housing and configured to support an operator of the utility vehicle; and
a sensor assembly including one or more sensors operable to sense a health characteristic of the operator.

37. The operator cab assembly of claim 36, wherein the chair includes a seat and a backrest coupled to the seat, and wherein the one or more sensors includes a first sensor coupled to one of the seat and the backrest.

38. The operator cab assembly of claim 37, wherein the first sensor is one of a temperature sensor, a proximity sensor, a pressure sensor, an oximetry sensor, a blood pressure sensor, an electrocardiogram sensor, and a heart rate sensor.

39. The operator cab assembly of claim 36, wherein the one or more sensors includes a first sensor supported by the housing such that the first sensor is oriented toward the chair, and wherein the first sensor is one of a camera and an infrared sensor.

40. The operator cab assembly of claim 36, further comprising a display device located in the housing, and wherein the display device is configured to display the health characteristic of the operator positioned on the chair.
